(19) United States
(12) Patent Application Publication
(10) Pub. No.: US 2014/0039296 A1
(43) Pub. Date: Feb. 6, 2014

(54) METHOD, SYSTEM AND CONTROL UNIT FOR A MONITORING-DEPENDENT IMAGING EXAMINATION

(71) Applicant: Marcus Wuebbe, Herzogenaurach (DE)

(72) Inventor: Marcus Wuebbe, Herzogenaurach (DE)

(21) Appl. No.: 13/957,749

(22) Filed: Aug. 2, 2013

(30) Foreign Application Priority Data
Aug. 3, 2012 (DE) 102012213765.3

(51) Int. Cl. A61B 6/00

(52) U.S. Cl. CPC A61B 6/541
USPC 600/413; 600/407

(57) ABSTRACT

In a method, control unit and system to acquire an image of a patient, possibly undergoing an examination according to a current examination plan with an imaging device, a vital function of the patient is monitored via a monitoring unit, and the overrun of a limit value by the monitored vital function is communicated to a determination unit by the monitoring unit, using the vital function, the determination unit selects a new examination plan that is suitable to detect a possible cause of the overrun of the vital function. An image of the patient is then acquired with the imaging device according to the new examination plan.

### FIELD OF THE INVENTION

The invention concerns a method to acquire an image of a patient, as well as a control unit to control an imaging device and a system, composed of a control unit and imaging device, for implementation of such a method.

### DESCRIPTION OF THE PRIOR ART

In diagnostic imaging methods, it is typical to monitor vital functions (for instance in general given patients with life-threatening illnesses) in order to be able to react quickly given degradation of the status. This is particularly true for examinations that require a longer period of time and also offer a poor direct access to the patient. In a magnetic resonance data acquisition procedure, the patient in the opening of the magnet is poorly accessible; movements and devices in the acquisition region also would interfere with the measurement. Not least, the use of various sensors is disrupted due to the strong magnetic field and the alternating electromagnetic fields. Conversely, due to the x-ray radiation that is used in computed tomography, the presence of other persons in the acquisition region is to be avoided in order to reduce their radiation exposure. Therefore, monitoring devices (for example display monitors) are used.

For example, from DE 10 2008 016286 A1 it is known to monitor a body function (such as respiration) and synchronize the image acquisition therewith in order to minimize artifacts in the acquired images due to the unavoidable movement.

From the product brochure for the Skyra product family of magnetic resonance tomography systems, a technology designated with DOT (“daily optimized throughput”) is known according to which suitable examination parameters of the imaging device are proposed for a predetermined examination plan of a patient in order to achieve an optimal result in a minimum amount of time.

However, if an acute health problem should occur in the patient during an examination in a system of the type in the cited prior art, which health problem is detected via the monitoring of a vital function, the patient is typically removed from the imaging device in order to be able to make a diagnosis conventionally and take such measures as may be necessary. If a new imaging of specific organs should be required, it is thus necessary to work out a new examination plan, and plan and implement a new examination.

### SUMMARY OF THE INVENTION

An object of the present invention is to provide a method and an imaging device that allow a diagnosis of a disruption of the vital functions to be made as effectively as possible.

A method according to the invention for the acquisition of an image of a patient includes a step for monitoring a predetermined vital function of the patient via a monitoring unit, while the patient is located in the acquisition region of an imaging device. Furthermore, the method includes a step of communication from the monitoring unit to a control unit of a deviation of the predetermined vital function from a predetermined desired value by more than a predetermined limit value. As a next step, a first examination plan is automatically determined by the control unit for the imaging device using the communicated deviation of the vital function, with the determination of the examination plan being executed by the control unit to cause an imaging examination to be implemented by the imaging device according to the examination plan that is designed to detect a possible cause of the deviation of the vital function. In a further step, an image of the patient is acquired by the imaging device according to the examination plan, as controlled by the control unit.

Upon communication of a deviation of a vital function of the patient in the imaging device that exceeds a limit value, the method according to the invention causes an examination plan to be determined that enables an immediate imaging examination of the patient who is still located in said imaging device. In this way the patient can also be examined for the cause of the deviation in the same examination session, without losing time. In comparison to a later, independent examination, the time for the preparation of the patient can also be saved, such that the imaging device is in operation for as long a period of time as possible without downtimes, so the imaging device is utilized more economically.

The invention also concerns a control unit to control an imaging device, which control unit is suitable for the execution of the above-described method according to the invention. The control unit has a first interface for communication with a monitoring unit to monitor a vital function of a patient and a second interface to control the imaging device. Moreover, the control unit has a determination unit or module that is configured to determine an examination plan, wherein the determination unit is designed to receive a communication about a deviation of a vital function from a predetermined desired region via the first interface for communication, and using said communication to determine an examination plan that is suitable to detect an optimal cause of the deviation of the vital function given execution by the imaging device.

Furthermore, the invention concerns a system that has such a control unit, the system furthermore having a monitoring unit. The monitoring unit has an interface for communication with the control unit and is designed to monitor a vital function of a patient and communicate deviations from a predetermined desired value by more than a predetermined limit value to the control unit via the interface.

The control unit according to the invention and the system according to the invention are designed to execute the method according to the invention and to thereby confer to the user the advantages that have already been described.

In a further embodiment, between the steps of determining an examination plan and the acquisition of an image the method has the steps: to output the selected first examination plan to an operator and to register an input of the operator, wherein the subsequent step of acquiring an image via the imaging device according to the first examination plan is only started by the control unit if the input of the operator signals an approval.

In this way it is possible for an operator to assess the proposed examination plan and, if applicable, to suppress the implementation of the additional image acquisition if the condition of the patient requires it, or if the proposed image acquisition does not appear to be promising. An unnecessary occupation of the imaging device can thus be prevented.

In a preferred embodiment of the method, the monitoring of the predetermined vital function takes place while the imaging device executes an image acquisition according to a second examination plan.

The monitoring of the vital functions preferably occurs during an image acquisition that takes place according to a second, pre-planned examination plan (with the above-discussed examination plan being a first examination plan). Since the image acquisition can take a longer time depending on the imaging apparatus and type of imaging method, a monitoring of the vital functions occurs directly given acutely ill patients. If one of the detected vital values deviates too significantly from the desired value, in the method according to the invention this is communicated to the control unit, and this immediately determines a suitable first examination plan for acquisition of a possible cause so that this image acquisition can take place immediately with the patient without additional preparation in order to determine the cause and appropriately care for the patient.

In an embodiment, the control unit according to the invention examination plans can be stored and accessed, with the determination unit designed to select a suitable examination plan from the stored examination plans.

In this way it is possible to prepare a suitable selection of examination plans for the control unit and to configure the determination unit more simply by associating selection criteria with each stored examination plan.

The control unit according to the invention can be configured to access stored examination plans via a network interface.

It is thereby possible for the control unit to access (for example) a centralized and comprehensive database or an expert system in which an examination plan that is suitable for the present disruption of the vital function is stored. In particular, it is possible for such an external source to correlate the examination plan with other data (for example patient data of the patient to be examined) so as to result in the most promising examination being then conducted.

In another embodiment, the determination unit is configured to access stored examination plans is a database. This database can be part of the control unit itself. It is then ensured that the control unit is able to promptly and reliably react to emergency situations, even without a network connection or without an active database on the network.

Furthermore, the system according to the invention can include an imaging device controlled by the control unit for the image acquisition.

It is thus advantageously ensured that the control unit and the imaging device can communicate and interact with one another smoothly.

The imaging device of the system can be a magnetic resonance (MR) tomography apparatus.

Magnetic resonance tomography allows imaging of nearly all organs in a multitude of different ways, such that there is a high probability that the imaging device will be able to detect a possible cause of the deviation of the vital function. An examination in a magnetic resonance tomography apparatus is particularly time-intensive and expensive, such that the acceleration of the MR examination via the method according to the invention produces a particularly significant cost advantage.

### BRIEF DESCRIPTION OF THE DRAWINGS

FIG. 1 schematically illustrates a first embodiment of a control unit according to the invention and a system according to the invention.

FIG. 2 schematically illustrates a second embodiment of a control unit according to the invention and a system according to the invention.

FIG. 3 is a flowchart of an embodiment of the method according to the invention.

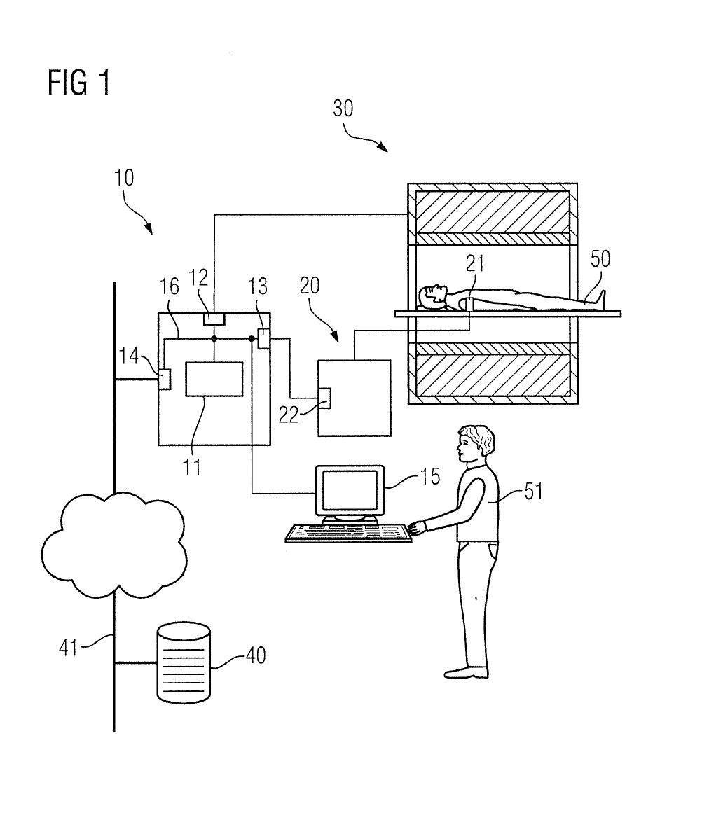

### DESCRIPTION OF THE PREFERRED EMBODIMENTS FIG. 1 is a schematic representation of a control unit 10, and a system that includes the control unit 10, a monitoring unit 20 and an imaging device 30.

To acquire an image according to a second examination plan, a patient 50 is located in the imaging device. For example, the imaging device 30 can be: an MR tomography apparatus; x-ray-based computed tomography apparatus or to generate ultrasound or PET images. The present invention is preferably provided in connection with an MR apparatus or a computed tomography apparatus, but alternative embodiments provide for other imaging methods to be used.

A sensor 21 is arranged on the patient 50 so as to detect a vital function of the patient and communicates a signal corresponding to the vital function to a monitoring unit 20. For example, the sensor can detect heart rate, blood pressure, oxygen saturation in the blood, EEG, EKG or other values. The type of sensor 21 is dependent on the status of the patient and the respective examination. For example, some sensors are not compatible with static magnetic fields and high-frequency radio waves of an MR tomography apparatus. The sensor 21 relays the signals wirelessly, via wires, or via other suitable transmission means (such as optical fibers or pressure conduits) to the monitoring unit 20.

The monitoring unit (often also designated as a monitor) receives the signals of the sensor 21 and determines from these signals a parameter indicative of a vital function of the patient. The monitoring unit 20 compares the determined value with predetermined limit values for the monitored vital parameters. Indicated as examples at the monitoring unit 20 in FIG. 1 are values for blood pressure and pulse. Upon the determined value exceeding an upper limit value or falling below a lower limit value, the monitoring unit 20 emits an alarm via an interface 22. The interface 22 can be an interface for simple signal relaying or a wireless connection via which a simple signal level or a complex data protocol is transmitted.

The control unit 10 has an interface 13 via which it is connected with the monitoring unit 20 via its interface 22 in order to receive the vital value alarm.

Furthermore, the control unit 10 is connected via an interface 12 with the imaging device 30 in order to control the imaging device 30 via this interface 12. The interface 12 can be of proprietary or standard design. Moreover, the control of the imaging device 30 concerns the regions of the patient 50 that are to be acquired in the image acquisition and the type of examination and its parameters. For example, for MR tomography the imaging device 30 can be controlled by the control unit 10 such that only the head of the patient 50 is imaged, and in particular such that the CSF should be shown. Such information for controlling an imaging examination is assembled into an examination plan in the control unit 10.

The control unit 10 moreover has a determination unit 11. For example, this determination unit 11 can be a processor that is connected via a processor bus 16 with the interfaces 12, 13, 14. If the determination unit 11 receives an alarm from the monitoring unit 20 via the interface 13, it evaluates the information included therein. In particular, an identification is made as to which vital parameters are affected and in which direction the limit value has been exceeded. The determination unit 11 determines a first examination plan (“first” in the sense of being different from the “second” examination plan) corresponding to this information. The first examination plan enables a detection by the imaging device 30 of a possible cause for such a limit value overrun. For example, given a sudden increase of the blood pressure and/or of the heart rate, an examination of the chest area can be reasonable in order to detect a disruption of the cardiac function as a possible cause. In FIG. 1, for this purpose the determination unit 1) accesses a database 40 (in which suitable examination plans are stored) via a network interface 14 and a network 41. However, it is also possible for the determination unit 11 to relay the information of the alarm notification to an expert system 40, which then communicates to the determination unit 11 a suitable examination plan via the network interface 14. The examination plans may already have suitable decision criteria associated therewith, by linking or embedding, by means of which a decision can be made.

In a preferred embodiment, the determination unit 11 presents the first examination plan to an operator 51 at an operating unit 15. Via the operating unit, the operator can then inform the determination unit 11 via an input as to whether the presented examination plan should be executed. For example, this can take place by pressing a button, clicking on a button or tapping a field on a touch-sensitive screen.

The control unit 10 then controls the imaging device 30 according to the first examination plan such that the possible cause for the limit value overrun of the vital parameter is detected in an image.

In a preferred embodiment, the operator can also suppress the execution of the first examination plan via an input.

In another embodiment of the invention, the execution takes place automatically without input by the operator 51.

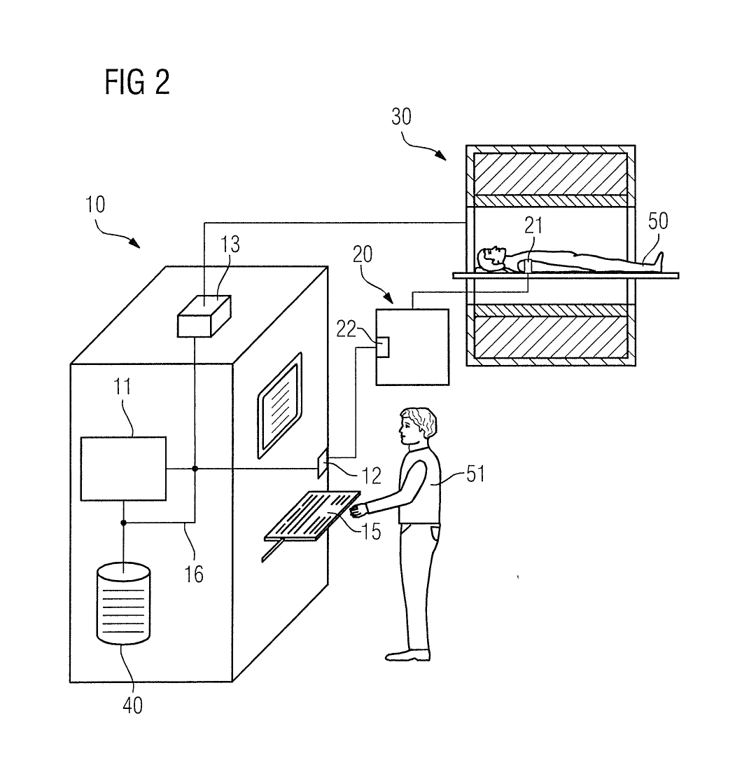

FIG. 2 shows a further embodiment of the control unit 10 according to the invention and of the system. Among other things, the database 40 and the operating unit 15 are integrated into the control unit of FIG. 2, such that a more compact design results. The system is therefore also independent of an external network 41 and databases that can be reached in said network 41, for which reason the availability in a mobile installation or even given disruptions in the network 41 is ensured.

It is also possible for the control unit 10 and the monitoring 20 to be integrated into the imaging device 30, such that a particularly compact and cost-effective design results.

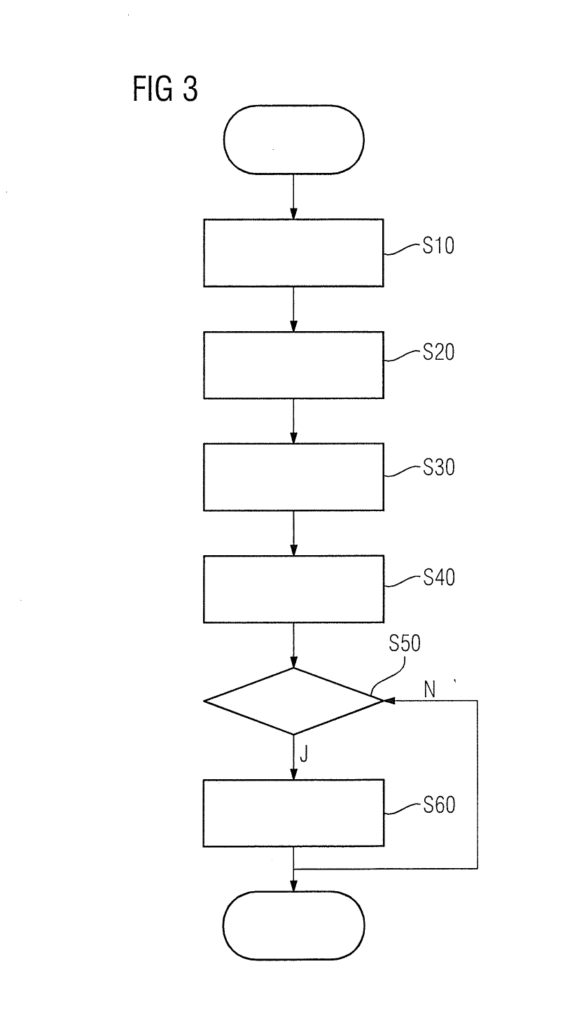

The individual steps of a method according to the invention are presented in FIG. 3.

Step S10 concerns the image acquisition of a patient via the imaging device 30 according to a second, planned examination plan, wherein a monitoring of the predetermined vital functions takes place via a monitoring unit 20 while the imaging device 30 executes an image acquisition (controlled by the control unit 10) according to a second examination plan.

In Step S20, the monitoring unit 20 communicates to the control unit 10 a deviation of the predetermined vital function from a predetermined desired value by more than a predetermined limit value.

In Step S30, a decision unit 11 of the control unit 10 determines a first examination plan for the imaging device 30 using the communicated deviation of the vital function, wherein the selection of the control unit 10 is such that an imaging examination according to the new examination plan is suitable to detect a possible cause of the deviation of the vital function.

In Step S40, the control unit 10 provides the selected first examination plan to an operator 51 via an operating unit 15.

In Step S50, the control unit 10 detects an input of the operator 51 at the operating unit 15. If the operator 51 approves the proposed first examination plan, an examination by the imaging device 30 is started by the control unit according to the first examination plan.

In Step S60, the imaging device 30 acquires an image of the patient 50 according to the first examination plan, with the imaging device 30 being controlled by the control unit 10.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to acquire a medical image of a patient, comprising:
while a patient is located in an image data acquisition region of a medical imaging device, monitoring a predetermined vital function of the patient using a monitoring unit:
from said monitoring unit, communicating, to a control unit, a deviation of the monitored predetermined vital function from a predetermined desired value by more than a predetermined limit value;
using the communicated deviation of the vital function communicated to said control unit to determine, in said control unit, an examination plan for operating said medical imaging device that is designed to detect a cause of said deviation of said vital function; and
controlling said medical examination device from said control unit to acquire a medical image of the patient with the medical imaging device by operating the medical imaging device according to the examination plan.

2. A method as claimed in claim 1 comprising, between determining said examination plan and acquiring said medical image:
presenting said examination plan in a humanly perceptible form at an interface of said control unit;
allowing an operator to enter and input into the control unit via said interface that indicates acceptance of said examination plan; and
operating said medical imaging device from said control unit according to said examination plan only if said input indicating acceptance of said examination plan has been made to said control unit via said interface.

3. A method as claimed in claim 1 wherein said examination plan is a first examination plan and comprising, from said control unit, operating said medical imaging device according to a second examination plan, different from said first examination plan, contemporaneously with said monitoring of said predetermined function, and switching operating of said medical imaging device from said second examination plan to said first examination plan to acquire said image of the patient.

4. A control unit that controls operation of a medical imaging device in which a patient is located, said control unit comprising:
a processor having a monitoring unit interfaced that receives, from a monitoring unit, a deviation of a monitored predetermined vital function, of a patient monitored by the monitoring unit, from a predetermined desired value by more than a predetermined limit value;
said processing being configured to use the communicated deviation of the vital function received by the processor to determine an examination plan for operating said medical imaging device that is designed to detect a cause of said deviation of said vital function; and
said processor having an examination device interface via which said processor is configured to control a medical examination device to acquire a medical image of the patient with the medical imaging device by operating the medical imaging device according to the examination plan.

5. A control unit as claimed in claim 4 comprising a source of stored examination plans, and wherein said processor is configured to access said source of examination plans to select said examination plan that is designed to detect said cause of said deviation of said vital function.

6. A control unit as claimed in claim 5 wherein said processor is configured to access said source of stored examination plans via a network interface.

7. A control unit as claimed in claim 5 wherein said source of stored examination plans is a database formed by an electronic memory accessible by said processor.

8. A medical imaging system comprising:
a monitoring unit adapted to monitor a vital function of a patient located in a medical imaging device;
a control unit;
said monitoring unit being configured to communicate, to said control unit, a deviation of the monitored predetermined vital function from a predetermined desired value by more than a predetermined limit value;
said control unit being configured to use the communicated deviation of the vital function communicated to determine an examination plan for operating a medical imaging device that is designed to detect a cause of said deviation of said vital function; and
said control unit being configured to control said medical examination device to acquire a medical image of the patient with the medical imaging device by operating the medical imaging device according to the examination plan.

9. A system as claimed in claim 8 wherein said system comprises said medical imaging device.

10. A system as claimed in claim 9 wherein said medical imaging device is a magnetic resonance tomography apparatus.
